(19) United States
(12) Patent Application Publication
(10) Pub. No.: US 2014/0088462 A1
(43) Pub. Date: Mar. 27, 2014

(54) ULTRASOUND NEUROMODULATION TREATMENT OF GASTROINTESTINAL MOTILITY DISORDERS

(71) Applicant: David J. Mishelevich, Playa del Rey, CA (US)

(72) Inventor: David J. Mishelevich, Playa del Rey, CA (US)

(21) Appl. No.: 13/623,880

(22) Filed: Sep. 21, 2012

(51) Int. Cl. A61N 7/00; A61N 2/08

(52) U.S. Cl. 601/2; 600/9

(57) ABSTRACT

Disclosed are methods and systems and methods for non-invasive neuromodulation using ultrasound to treat gastrointestinal motility disorders. The neuromodulation can produce acute or long-term effects. The latter occur through Long-Term Depression (LTD) and Long-Term Potentiation (LTP) via training. Included is control of direction of the energy emission, intensity, frequency, pulse duration, pulse pattern, and phase/intensity relationships to targeting and accomplishing up regulation and/or down regulation.

### CROSS REFERENCE TO RELATED APPLICATIONS

This patent application claims priority to Provisional Patent Application Numbers 61/537,881 filed Sep. 22, 2012, entitled “ULTRASOUND NEUROMODULATION TREATMENT OF GASTROINTESTINAL MOTILITY DISORDERS.”

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Neuromodulation including one or more ultrasound sources for neuromodulation of gastrointestinal regions to up-regulate or down-regulate neural activity for the treatment of a medical condition.

### BACKGROUND OF THE INVENTION

It has been demonstrated that focused ultrasound directed at neural structures can stimulate those structures. If neural activity is increased or excited, the neural structure is up regulated; if neural activated is decreased or inhibited, the neural structure is down regulated. The potential application of ultrasonic therapy of neural structures has been suggested previously (Gavrilov L. R., Tsirulnikov E. M., and I. A. Davies, “Application of focused ultrasound for the stimulation of neural structures,” Ultrasound Med Biol. 1996;22(2):179-92. and S. J. Norton, “Can ultrasound be used to stimulate nerve tissue?,” BioMedical Engineering OnLine 2003, 2:6). Norton also noted that monophasic ultrasound pulses are more effective than biphasic ones.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested. The above approach is incorporated in a patent application submitted by Tyler (Tyler, William, James P., PCT/US2009/050560, WO 2010/009141, published Jan. 21, 2011).

Alternative mechanisms for the effects of ultrasound may be discovered as well. In fact, multiple mechanisms may come into play, but, in any case, this would not effect this invention.

Because of the utility of ultrasound in the neuromodulation of deep neural structures, it would be both logical and desirable to apply it to the treatment of gastrointestinal motility disorders.

### SUMMARY OF THE INVENTION

It is the purpose of this invention to provide methods and systems for non-invasive neuromodulation using ultrasound to treat gastrointestinal motility disorders, including constipation and diarrhea. It can also be used to treat gastrointestinal-system cramping, including reducing the constriction of GI ducts such as the bile duct and the duct to the gall bladder. Application of the ultrasound neuromodulation can be on the external surface of the body and/or within the GI tract. Such neuromodulation can produce acute effects or Long-Term Potentiation (LTP) or Long-Term Depression (LTD). Included is control of carrier frequency, neuromodulation frequency, direction of the energy emission, intensity, pulse pattern, pulse duration, and phase/intensity relationships to targeting and accomplishing up-regulation and/or down-regulation. Use of ancillary monitoring or imaging to provide feedback is optional. In embodiments where concurrent imaging is performed, the device of the invention is constructed of non-ferrous material. An application of the invention is to accelerate the carriage of the output of the contents of the stomach through the small intestine where absorption of nutrients occurs so less such absorption occurs and the therapeutic malabsorption fosters weight loss. Another application is the prediction of those patients who will have GI motility disorders associated with radiotherapy.

Multiple gastrointestinal targets can be neuromodulated singly or in groups to treat motility disorders. To accomplish the treatment, in some cases the neural targets will be up regulated and in some cases down regulated, depending on the given neural target. Targets have been identified by such methods as electrogastography or imaging.

In some cases neuromodulation will be bilateral and in others unilateral. The specific targets and/or whether the given target is up regulated or down regulated, can depend on the individual patient and relationships of up regulation and down regulation among targets, and the patterns of stimulation applied to the targets.

### BRIEF DESCRIPTION OF THE DRAWINGS

FIG. 1 shows ultrasound-transducer neuromodulation of the large intestine using an internal pill-style version trailed by a power-control unit.

FIG. 2 demonstrates neuromodulation of the small intestine using an external ultrasound transducer.

FIG. 3 shows a block diagram for a control of the neuromodulation based on feedback as to level of gastrointestinal motility, either in auto-tune mode or patient-feedback mode.

FIG. 4 shows a block diagram of the control circuit for ultrasound neuromodulation variables.

### DETAILED DESCRIPTION OF THE INVENTION

It is the purpose of this invention to provide methods and systems and methods for neuromodulation of abdominal and/or pelvic targets using therapeutic ultrasound to treat gastrointestinal motility disorders. The invention is applicable to animals as well as people. Application of the ultrasound neuromodulation can be on the external surface of the body and/or within the GI tract. Such neuromodulation systems can produce applicable acute or long-term effects. The latter occur through Long-Term Depression (LTD) or Long-Term Potentiation (LTP) via training. Thus, for example, it is possible to treat constipation in the first by acute treatments as needed and through training achieved through repeated such treatments have a long result of decrease in or elimination of constipation. Included is control of direction of the energy emission, intensity, frequency, pulse duration, pulse pattern including pulse rate, and phase/intensity relationships for ultrasound-beam steering and/or mechanically redirecting the position and/or direction of ultrasound beams to targeting and accomplishing up-regulation and/or down-regulation.

Ultrasound is acoustic energy with a frequency above the normal range of human hearing (typically greater than 20 kHz). In this invention, ultrasound-neuromodulation techniques refers to the delivery of ultrasound energy to tissue in the brain, spinal cord, or other structures having an acoustic frequency in a range of 0.3 MHz to 0.8 MHz with acoustic intensity greater than 20 mW/cm² at the target tissue. The frequency in the range of 0.3 MHz to 0.8 MHz represents the carrier frequency on which amplitude modulation is applied. The amplitude modulation frequency for inhibition or down regulation is typically lower than 500 Hz (depending on condition and patient). The amplitude modulation frequency for excitation is typically in the range of 500 Hz to 5 MHz again depending on condition and patient. In one embodiment, the modulation frequency of lower than approximately 500 Hz is divided into pulses 0.1 to 20 msec. repeated at frequencies of 2 Hz or lower for inhibition or down regulation. In one embodiment, the amplitude modulation frequency of higher than approximately 500 Hz. is divided into pulses 0.1 to 20 msec. repeated at frequencies higher than 2 Hz for up regulation. In some embodiments the acoustic intensity is greater than about 30 mW/cm² at the target tissue. The acoustic intensity is less than the appropriate target- or patient-specific levels at which no tissue damage is caused. Ultrasound therapy can be combined with therapy using other devices (e.g., Transcranial Magnetic Stimulation (TMS)).

In the gastrointestinal tract down regulation is used to treat diarrhea and up regulation used to treat constipation. Ultrasound therapy can be combined with therapy using other therapies such as drugs.

The lower bound of the size of the spot at the point of focus will depend on the ultrasonic frequency, the higher the frequency, the smaller the spot. In treating the gastrointestinal tract, a high degree of focus is not required or applied. Ultrasound-based neuromodulation operates preferentially at low frequencies relative to say imaging applications so there is less resolution. As an example, Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. In any case, an ultrasound conduction medium will be required to fill the space between the face of the ultrasound transducer or ultrasound lens and the surface of the skin.

Gastrointestinal activity can be assessed objectively by myoelectric activity, measurement of pressure changes, and detection of motion, say by movement of accelerometers. Such sensors can be built in to a neuromodulation device passing through the GI tract, can be placed in a separate sensing device passing through or inserted into the GI tract, or for myoelectric signals can be detected by sensors external to the body such as myoelectric signals captured by electrodes placed on the skin.

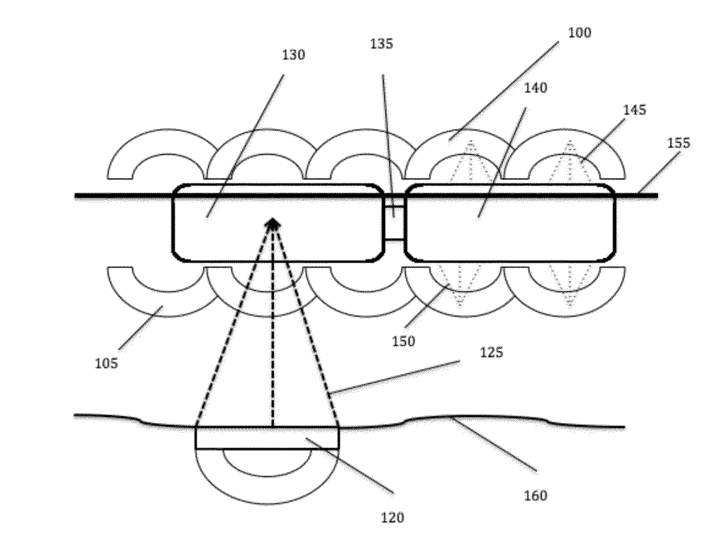

FIG. 1 shows gastrointestinal lumen 100 within body 145. The ultrasound neuromodulation is generated by ultrasound transducer capsule 140 with ultrasonic beam 145 hitting one side of the lumen and ultrasonic beam 150 hitting the other. In fact, the beams generated will be 360 degrees around the transducer and longitudinal along the length of the transducer. Power to the ultrasound transducer is provided from power-supply capsule 130 through connection 135. Power-supply capsule 130 could contain a battery allowing low-power stimulation by ultrasound transducer 140, but in most embodiments will contain an antenna and power transducer. The electromagnetic energy source 120 is connected to a higher-level power source with power control, not shown. The output of electromagnetic energy source 120 (e.g., Witricity) is beam 125 whose power is absorbed by power-supply capsule 130. The activity of the lumen can be monitored in some cases by probe 155, either for determination of neuromodulation variables, or for real-time feedback. Examples of physiological feedback measurement are internal electrodes, electronic pressure transducers, or manometic instrumentation. Endoscopically placed probe 155 is connected to the monitoring instrumentation (not shown) by a cable, also not shown. In another embodiment (not shown), the sensors (e.g., myoelectric sensors or pressure sensors) are built into the ultrasonic transducer 140 and/or power-supply capsule 130. Data may be collected continuously or between pulses or between pulse trains. In another embodiment, electrodes on the surface of the body of the patient detect the myoelectric activity of the colon. While the FIG. 1 refers to the colon, the invention applies to the colon as well.

Transducer array assemblies of this type may be supplied to custom specifications by Imasonic in France (e.g., large 2D High Intensity Focused Ultrasound (HIFU) hemispheric array transducer)(Fleury G., Berriet, R., Le Baron, 0., and B. Huguenin, “New piezocomposite transducers for therapeutic ultrasound,” 2^{nd} International Symposium on Therapeutic Ultrasound—Seattle—July 31-Aug. 2, 2002, typically with numbers of ultrasound transducers of 300 or more. Keramos-Etalon in the U.S. is another custom-transducer supplier. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

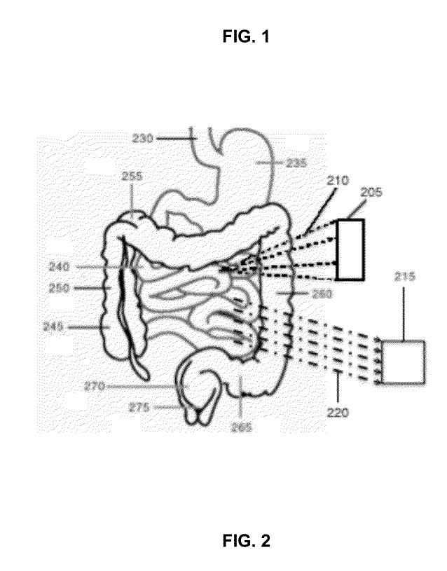

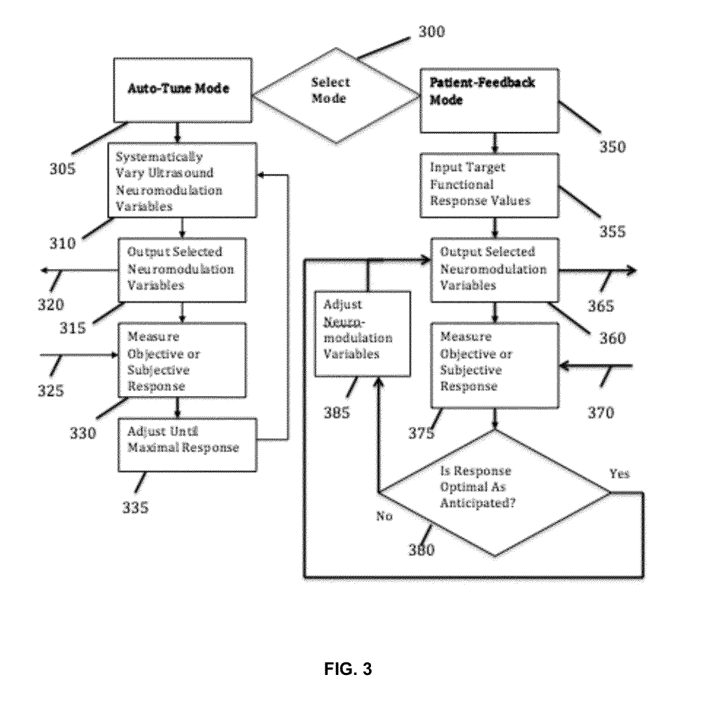

FIG. 2 shows gastrointestinal organs that could be neuromodulated including the esophagus 230, the stomach 235, the small intestine 240, the cecum 245, the ascending colon, 250, the transverse colon 255, the descending colon 260, the sigmoid colon 265, the rectum 270, and the anus 275. An additional target is the vagal nerve. Ultrasound transducer 205 with its ultrasound beam 210 (shown neuromodulating the small intestine) provides neuromodulation. Other embodiments include multiple ultrasound transducers focusing on one or more targets. The signals indicating level of gastrointestinal motility (e.g., by electrogastroenterogram) is detected by sensor 215. The control diagram for taking this feedback and controlling the level of neuromodulation is shown in FIG. 3. FIG. 2 illustrates the internal view of the body, but each ultrasound transducer will be applied to the skin of the body (not shown). For ultrasound to be effectively applied to the external body surface and transmitted to and through the body, coupling must be put into place. Ultrasound transmission (for example Dermasol from California Medical Innovations) medium placed, if applicable, within the ultrasound transducer cavity so that a contiguous surface is presented to the surface of the skin. This is true whether the ultrasound transducer is applied to the anterior surface of the abdomen, and/or one or both surfaces of the abdomen, and/or the back, and or the surface surrounding the rectum. This contiguous surface is not sufficient, however. To “complete the circuit,” in FIG. 2, an ultrasound-conduction gel layer (not shown) is placed between ultrasound transducer/lens 205 and the surface of the body (not shown). In other embodiments, multiple ultrasound transducers whose beams intersect at that target replace an individual ultrasound transducer for that target. In other embodiments, both internal and external ultrasound transducers provide neuromodulation.

With respect to the control of motility of the small intestine as could be done as in either FIG. 1 or FIG. 2, there can be acceleration of the carriage of the output of the contents of the stomach through the small intestine where absorption of nutrients occurs so less such absorption occurs and the therapeutic malabsorption fosters weight loss. An additional approach is the use of tagged food or drugs combined with imaging to judge the results.

FIG. 3 shows a block diagram for a control of the neuromodulation based on feedback as to level of gastrointestinal motility. A key element for effective neuromodulation is to tune it to the specific patient at the specific time of treatment. As shown in FIG. 3 in Select Mode 300, two modes are available, Auto-Tune Mode 305 and Patient-Feedback Mode 350. Auto-Tune Mode 305 is used when the ultrasound neuromodulation is being initially set up for the particular patient. Patient-Feedback Mode 350 is used during the subsequent treatment sessions.

In Auto-Tune Mode 305, the neuromodulation variables (carrier frequency, neuromodulation frequency, transducer direction, intensity, pulse pattern including pulse rate, pulse duration, intensity, and phase/frequency relationships for ultrasound-beam steering and/or mechanically redirecting the position and/or direction of ultrasound beams) are varied, not necessarily all in a given session. Hill climbing or other optimization algorithms are used for optimization. Neuromodulation at the current set of variable values is output via block 315 through output channel 320. The physiological evidence of bowel activity (e.g., via electrogastrography (electrogastrogram, EGG) or electrocologram (intra-colonic recording (see FIG. 1) or external recording from external cutaneous electrodes) or subject patient-report results come back through channel 325 and measured in block 330. Based on whether maximal response has been achieved as judged in block 335, control is exercised to either maintain the current values if the response has been judged as satisfactory or to vary the neuromodulation variables in 310 if not. In some implementations, only objective feedback is used and patient feedback is not utilized.

In the Patient-Feedback Mode 350 of FIG. 3, the treatment planner inputs target functional response values in 355 resulting in the output of the selected neuromodulation variables in 360 through output channel 365. The objective response and the subjective input (e.g., level of feeling or motility or hearing gurgling) come back through channel 370 in Measure Objective or Subjective Response 375 where subsequently the response is evaluated in block 380 (“Is Response Optimal As Anticipated?”). If the response is optimal, then the neuromodulation variables are left as they were; if the response is not optimal, the variables are adjusted in 385 and output via block 360 through output channel 365. One embodiment of the mode is to provide the patient the capability of turning the level of motility up or down, including when sitting on a toilet. In some implementations, only subjective feedback is used and objective feedback is not utilized. Motility feedback can be obtained from surface electrodes detecting myoelectric activity, internal electrodes inserted into the GI tract, imaging (likely ultrasound imaging), internal pressure sensors, or other suitable means. The latter can include using one or more microphones to detect evidence of motility such as gurgling or detection of releasing of fluid through the wall of the gut. Electromyographic activity can be obtained using an electrogastroenterogram for the small intestine or the stomach (an electrogastrogram is used for the stomach alone), and an electrocologram for the large intestine. Maximum muscle contraction rates in waves per minute are approximately three for the stomach, 12 for the duodenum, 8 for the ileum, 11 for the jejunum, and 17 for the rectum. The use of electromyography, electrogastrography, and imaging of one form or another can not only be used for feedback-control purposes and tuning, but also to see how well the neuromodulation is working by looking inside the body and seeing its impact on the GI-system components.

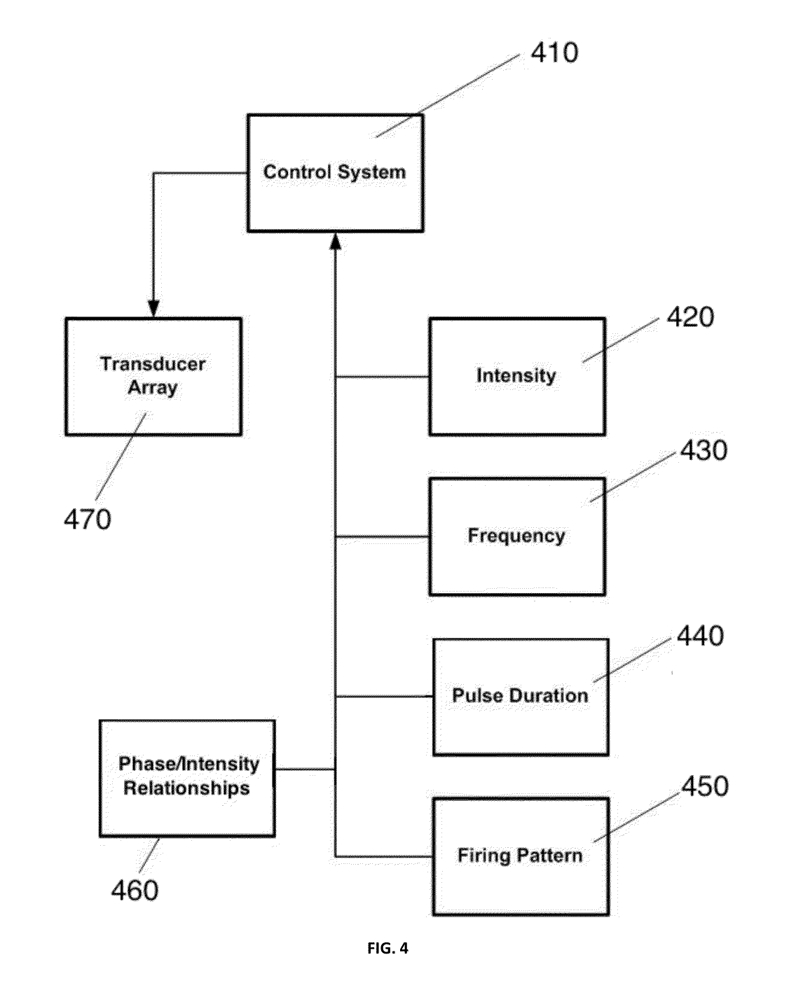

FIG. 4 shows an embodiment of a control circuit. The positioning and emission characteristics of transducer array 470 are controlled by control system 410 with control input with neuromodulation characteristics determined by settings of intensity 420, frequency 430 (can be carrier and/or neuromodulation frequency), pulse duration 440, firing pattern 450, and phase/intensity relationships 460 for beam steering and focusing on neural targets. Instead of phase/frequency relationships that can steer the ultrasound beam, 460 can represent mechanically altering the direction of the ultrasound beam, including axial or radial mechanical perturbations of the ultrasound transducers.

The use of electromyography, electrogastrography, and imaging of one form or another can not only be used for feedback-control purposes and tuning, but also to see how well the neuromodulation is working by looking inside the body and seeing its impact on the GI-system components.

In still other embodiments, ultrasound neuromodulation is combined with one or more excitatory or inhibitory agents such as drugs, pulsed magnetic stimulation, direct electrical stimulation, or traditional remedies such as enemas.

The invention allows stimulation adjustments in variables such as, but not limited to, carrier frequency, neuromodulation frequency, transducer direction, intensity, pulse pattern including pulse rate, pulse duration, intensity, and phase/frequency relationships for ultrasound-beam steering and/or mechanically redirecting the position and/or direction of ultrasound beams.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method of deep-brain neuromodulation using ultrasound stimulation, the method comprising:
a. aiming at least one ultrasound transducer at least one gastrointestinal target, and
b. energizing at least one transducer to deliver pulsed ultrasound energy to the at least one target,
whereby the gastrointestinal motility disorders are treated.

2. The method of claim 1 wherein the motility conditions treated are selected from the group consisting of constipation and diarrhea.

3. The method of claim 1 wherein the carriage of the contents of the stomach through the small intestine is accelerated such that malabsorption occurs and weight loss achieved.

4. The method of claim 3 where tagged substances combined with images are used to judge the results.

5. The method of claim 1 further comprising aiming an ultrasound transducer neuromodulating neural targets in a manner selected from the group of up-regulation for treating constipation, down-regulation for treating diarrhea.

6. The method of claim 1 wherein the effect is chosen from the group consisting of acute, Long-Term Potentiation, and Long-Term Depression.

7. The method of claim 1 wherein the ultrasonic neuromodulation is applied in a manner selected from one or a more of internal and external.

8. The method of claim 1 wherein one or a plurality of targets are selected from the group consisting of stomach, small intestine, vagal nerve, large intestine, and rectum.

9. The method of claim 1 wherein aiming comprises aiming a plurality of ultrasonic transducers to produce beams which intersect at a target.

10. The method of claim 1 wherein the ultrasound energy has a carrier frequency is in the range of 0.3 MHz to 0.8 MHz.

11. The method of claim 1 wherein the ultrasound energy is delivered at a power less than that causing tissue damage.

12. The method of claim 1 wherein the ultrasound energy has a stimulation frequency of lower than 500 Hz is applied for inhibition of neural activity.

13. The method of claim 12 wherein the ultrasound energy has a pulse duration in the range from 0.1 to 20 msec repeated at frequencies of 2 Hz or lower for down regulation.

14. The method of claim 1 wherein the ultrasound energy has a stimulation frequency for excitation is in the range of 500 Hz to 5 MHz.

15. The method of claim 14 wherein the ultrasound energy has a pulse duration in the range from 0.1 to 20 msec repeated at frequencies higher than 2 Hz for up regulation.

16. The method of claim 1 wherein the ultrasound has a focus area diameter in the range from is 0.5 to 150 mm.

17. The method of claim 1 further comprising applying mechanical perturbations radially or axially to move the ultrasound transducers.

18. The method of claim 1 wherein a feedback mechanism is applied, wherein the feedback mechanism is selected from the group consisting of myoelectric signals, pressure sensors, accelerometer sensors, ultrasound imaging, acoustic monitoring, and patient report.

19. The method of claim 1 wherein feedback is applied in a mode selected from the group consisting of auto-tune and patient feedback.

20. The method of claim 1 wherein ultrasound therapy is combined with or replaced by one or more therapies selected from the group consisting of Transcranial Magnetic Stimulation (TMS), direct electrical stimulation (DBS), Radio-Frequency (RF) therapy, enemas, and medications.
